(19) United States
(12) Patent Application Publication
(10) Pub. No.: US 2020/0276537 A1
(43) Pub. Date: Sep. 3, 2020

(54) WASTE GAS EMISSION CONTROL SYSTEM

(71) Applicant: Johnson Matthey Public Limited Company, London (GB)

(72) Inventor: Henrik BOMAN, Perstorp Industripark (SE)

(21) Appl. No.: 16/649,851

(22) PCT Filed: Oct. 3, 2018
(86) PCT No.: PCT/GB2018/052817

§ 371 Date: Mar. 23, 2020

(30) Foreign Application Priority Data
Oct. 3, 2017 (GB) 1716132.4

(51) Int. Cl. B01D 53/75; B01J 8/00; C07C 45/38

(52) U.S. Cl. CPC B01D 53/75; B01J 8/008; C07C 47/04; C07C 2523/881; C07C 45/38

(57) ABSTRACT

A process for the production of formaldehyde is disclosed. The process comprises feeding a feed stream comprising methanol to a reactor; converting the methanol to formaldehyde in the reactor using a mixed oxide catalyst to produce a process stream comprising formaldehyde; separating formaldehyde from the process stream to create a product stream comprising formaldehyde and a waste gas stream; feeding at least part of the waste gas stream to a steam condenser to raise the temperature of the at least part of the waste gas stream to create a heated waste gas stream; and feeding the heated waste gas stream to a catalytic combustion bed to catalytically combust components of the heated waste gas stream to create a combusted waste gas stream.

### FIELD OF INVENTION

The present invention concerns an emissions control system for the catalytic combustion of components of a process waste gas stream. In particular, but not exclusively, the present invention concerns an emissions control system for use in a process for the production of formaldehyde, for example as formalin or UFC. The present invention also concerns a process for the production of formaldehyde, for example as formalin or UFC.

### BACKGROUND

Formaldehyde can be produced by the catalytic oxidative dehydrogenation of methanol. Processes for carrying out such production are known, for example from WO9632189 or US2504402. The catalyst typically comprises a so-called “mixed oxide” catalyst comprising molybdenum and iron oxides. A well-known process for the production of formaldehyde is the Formox process offered by Johnson Matthey. The Formox process involves catalytic oxidative dehydrogenation of methanol over a mixed oxide catalyst. The Formox process is illustrated in FIG. 1. Methanol is mixed with air, vaporised and fed as a feed stream to a reactor, where it is converted into formaldehyde. The process stream leaving the reactor is passed to an absorber and the formaldehyde is removed from the process stream and exits in a product stream at the bottom of the absorber, typically as formalin or UFC. A part of the waste gas stream from the top of the absorber is fed to an emissions control unit (the rest, for example, being recycled), where hazardous components such as carbon monoxide, DME and methanol, are burned by catalytic combustion to produce a combusted waste gas stream that can be vented via a stack. In the present design, the combusted waste gas stream is used to pre-heat the waste gas stream entering the emissions control system to the required ignition temperature for the catalytic combustion. The present emissions control system offers significant advantages over processes without such a system, but it is desirable to seek to improve the system further so as to reduce capital costs and reduce pressure drops. That is particularly the case since emission control systems may be retro-fitted onto existing processes to improve their emissions standards.

The present invention seeks to provide an improved emissions control system and process for the production of formaldehyde.

### SUMMARY OF THE INVENTION

According to a first aspect of the invention there is provided a process for the production of formaldehyde, the process comprising:

feeding a feed stream comprising methanol to a reactor;

converting the methanol to formaldehyde in the reactor using a mixed oxide catalyst to produce a process stream comprising formaldehyde;

separating formaldehyde from the process stream to create a product stream, comprising formaldehyde, and a waste gas stream;

feeding at least part of the waste gas stream to a steam condenser to raise the temperature of the at least part of the waste gas stream to create a heated waste gas stream; and

feeding the heated waste gas stream to a catalytic combustion bed to catalytically combust components of the heated waste gas stream to create a combusted waste gas stream.

By feeding the waste gas stream to a steam condenser to raise the temperature of the waste gas stream, the temperature of the heated waste gas stream as it is fed to the catalyst bed can be controlled so as to maintain a constant temperature of the heated waste gas stream entering the catalyst bed. The control is more straightforward than prior art systems where the combusted waste gas stream is used to heat the incoming waste gas stream since the steam can be controlled independently. For example, in order to maintain a certain temperature into the catalyst bed, a minimum required amount of steam with a minimum required steam pressure may be used. For example, a steam pressure of 14.6 barg corresponds to a steam temperature of 200° C. and a steam pressure of 22.2 barg corresponds to a steam temperature of 220° C. In an example process, the catalyst bed contains a catalyst comprising PPd and PPt catalysts available from Johnson Matthey Formox and operates with 12 barg steam pressure. That pressure advantageously corresponds to a minimum export steam pressure from a typical plant. The steam temperature will typically need to exceed the inlet temperature of the catalyst bed by an approach temperature, to allow for effective heat exchange. In some embodiments therefore, the steam condenser is preferably fed with steam at a pressure of from 10 to 25 barg, preferably from 15 to 25 barg and most preferably from 17 to 20 barg. Such steam pressures may efficiently heat the waste gas stream. Steam condensate may be collected and reused. Efficient heat transfer in the steam condenser may also permit lower pressure drops as the waste gas passes through the steam condenser. Reducing pressure drops may be advantageous in providing a cost- and energy-efficient process as the energy required to compress the gases entering the process, and the cost of that energy, may be significant. Preferably the steam condenser and the catalyst bed are contained within a single vessel. Such an arrangement reduces the need for connecting pipework and may thus further reduce the pressure drop of the system.

The product stream comprising formaldehyde is preferably formalin or UFC. The mixed oxide catalyst preferably comprises molybdenum and iron oxides. The conversion of the methanol to formaldehyde in the reactor and the separation of the product stream comprising formaldehyde may, for example, be carried out according to the Formox process.

Preferably the waste gas stream enters in the region of the bottom of the steam condenser and flows up through the steam condenser and the heated waste gas stream flows up through the catalyst bed. That may have several advantages. For example, the catalyst bed in an emissions control system is typically supported on a catalyst net. Flowing the heated waste gas stream up through the catalyst bed means that the net is at the cooler, inlet end of the catalyst bed. That is advantageous as the net does not have to withstand as high temperatures and the risk of the net failing is reduced. It may thus be more straightforward to provide a net having the required mechanical strength. A further net may be required at the top of the catalyst bed to reduce movement of the catalyst due to the upward heated waste gas flow, however that net is not subjected to the same forces as the net at the bottom of the catalyst bed and thus does not need to be as strong. It can therefore be more easily designed to handle the high temperatures at the exit of the catalyst bed. Furthermore, flowing the waste gas stream in a single direction through the steam condenser and then onward through the catalyst bed may advantageously reduce pressure drops. Flowing the waste gas stream upwards may be particularly advantageous as condensate (for example, water) condensing in the steam condenser can then be allowed to flow downwards under gravity in a counter-current manner so that the hottest temperature is at the top of steam condenser where the heated waste gas stream passes to the catalyst bed. Preferably therefore steam enters the steam condenser in the region of the top of the steam condenser and flows down through the steam condenser, condensing to form a condensate and the condensate exits the steam condenser in the region of the bottom of the steam condenser.

Preferably the steam condenser is a shell and tube steam condenser and the waste gas stream flows through the tube side of the steam condenser and steam condenses in the shell side of the steam condenser. Such an arrangement may optimise pressure drop and heat transfer efficiency.

Preferably the process further comprises:

Feeding the combusted waste gas stream to a steam generator wherein the combusted waste gas stream is cooled and steam is produced.

Thus, the process may use the heat from the combustion to generate steam that can be used elsewhere in the process or elsewhere in the plant. For example, the steam could be provided to a plant steam net. While providing the steam to a plant steam net, and using steam from elsewhere in the plant in the steam condenser may be an efficient option if different pressures of steam are used in the steam condenser and generated in the steam generator, in a particularly preferred embodiment the steam produced in the steam generator is used in the steam condenser. Thus, the process preferably further comprises:

Feeding the steam from the steam generator to the steam condenser to raise the temperature of the waste gas stream in step d.

Thus the heat is recovered from the combusted waste gas stream and used to heat the waste gas stream before it is fed to the catalyst bed, but that recovery and heating is done indirectly using steam. The steam is produced using the heat from the combusted waste gas stream and is then used to transfer that heat to the incoming waste gas stream. An advantage of such a system is that the pressure drops on the steam side of the process do not affect the overall pressure drops of the formaldehyde production process. Thus, the effectiveness of the heat transfer on the steam side can be optimised without needing to account for the effect of any pressure drop on the overall formaldehyde process. The heat transfer may in any case be more efficient in a condenser than in a gas-gas heat exchanger as might be used if heating the waste gas stream with the combusted waste gas stream directly. Moreover, the heat integration can be more efficiently balanced as additional steam can be added if more heat is needed, or some steam can be removed and used elsewhere if there is an excess of heat. The system may also have advantages on start up since steam from another source can be used initially to heat the waste gas stream. That may remove the need to incur the cost of an electrical heater to heat the emissions control system on start-up.

Preferably the steam generator is a shell and tube steam generator and the combusted waste gas stream flows through the tube side of the steam generator and steam is generated in the shell side of the steam generator. Advantageously, that may reduce the pressure drop of the combusted waste gas stream. In some embodiments, the steam generator may comprise a steam super-heater and in some embodiments the steam generator may be a steam super-heater.

Preferably the steam condenser, the catalyst bed and the steam generator are contained within a single vessel. That may advantageously remove pressure drops that would be associated with connections between separate vessels. It may also provide a single unit that can be retrofitted to an existing plant. Containing the steam condenser, the catalyst bed and the steam generator within a single vessel may also be advantageous mechanically as it may eliminate the need for high-temperature piping and flanges that would otherwise be needed, particularly between the catalyst bed and the steam generator. The combusted waste gas stream leaving the catalyst bed in prior art systems may reach temperatures of around 550° C. and any piping and flanges between the catalyst bed and steam generator may therefore need to handle such temperatures. The temperature of the combusted waste gas stream leaving the steam generator may be around 230° C.-245° C. if, for example, 22.2 barg 220° C. steam is generated in the steam generator. Thus, if the catalyst bed and the steam generator are contained within the same vessel, the pipework and flanges of that vessel can be designed for temperatures of around 230° C.-245° C., instead of 550° C., which may result in significant savings. Moreover, when there is no need for piping and flanges between the catalyst bed and the steam generator in the present invention, the process temperature at the exit of the catalyst bed can advantageously be increased, for example to at least 580° C., preferably to at least 590° C. and more preferably to at least 600° C., at reasonable cost. Such an increase may improve the emissions control of the process. Preferably the steam generator produces steam having a pressure of from 10 to 25 barg, more preferably 15 to 25 barg. The steam generator may produce steam having a pressure of from 17 to 20 barg.

Preferably the steam condenser is a shell and tube steam condenser, wherein the waste gas stream flows through the tube side of the steam condenser and steam condenses in the shell side of the steam condenser; and the steam generator is a shell and tube steam generator, wherein the combusted waste gas stream flows through the tube side of the steam generator and steam is generated in the shell side of the steam generator. Keeping the waste gas stream and the combusted waste gas stream (that is, the process waste gas streams) on the tube side of the steam condenser and steam generator can have significant advantages for scaling up the emissions control system. In such a system, pressure drop can be maintained when scaling the system up by scaling the numbers of tubes with the capacity demand. That is desirably advantageous over prior art systems where the combusted waste gas stream is on the shell side and the waste gas stream is on the tube side and scale up is more complex.

Preferably, before being fed to the steam generator, the combusted waste gas stream is fed through an expander part of a turbocharger to drive a compressor part of the turbocharger in order to pressurise an air stream fed to the process to form part of the feed stream. Using at least some of the energy from the combusted waste gas stream in a turbocharger used to pressurise an air stream fed to the process to form part of the feed stream, and thus to pressurise the feed stream, is advantageously an efficient way to recover as much energy as possible from the combusted waste gas stream. Feeding the combusted waste gas stream to the turbocharger before feeding the combusted waste gas stream to the steam generator may be advantageous in making the best use of the high temperature combusted waste gas stream leaving the catalyst bed.

According to a second aspect of the invention there is provided an emissions control system for the catalytic combustion of components of a process waste gas stream, the emissions control system comprising: a catalyst bed comprising a catalyst for the catalytic combustion of the components of the process waste gas stream; and a steam condenser having a tube side in fluid communication with a process waste gas stream inlet and the catalyst bed, and a shell side in fluid communication with a steam inlet and a condensate outlet, such that, in operation, a process waste gas stream entering the process waste gas stream inlet is heated in the steam condenser before passing to the catalyst bed.

Preferably the emissions control system comprises a vessel containing both the catalyst bed and the steam condenser. Both the catalyst bed and the steam condenser being in the same vessel may advantageously result in a less expensive apparatus and an apparatus with an advantageously low pressure drop across the emission control system.

Preferably the process waste gas stream inlet is in the region of the bottom of the vessel; the tube side of the steam condenser comprises tubes, preferably vertical tubes, having an inlet end lower than an outlet end; the steam inlet is in the region of the top of the shell side of the steam condenser; the condensate outlet is in the region of the bottom of the steam condenser; and the catalyst bed is arranged above the steam condenser, such that, in operation, the process waste gas stream entering the process waste gas stream inlet flows up through the tube side of the steam condenser and up through the catalyst bed, and steam entering the steam inlet flows down through the shell side and condenses to form a condensate, wherein the condensate flows down through the shell side and out through the condensate outlet. Such an apparatus may be particularly efficient to operate and control, for example by controlling the level of condensate in the shell side.

Preferably the emissions control system further comprises a steam generator having a tube side in fluid communication with the catalyst bed and a process waste gas stream outlet, and a shell side in fluid communication with a boiler feed water inlet and a steam outlet, such that, in operation, the process waste gas stream leaving the catalyst bed is cooled in the steam generator, converting boiler feed water entering through the boiler feed water inlet into steam exiting through the steam outlet, before exiting the process waste gas stream outlet. The steam outlet may be connected to a plant steam net to export steam to the plant. Preferably the steam outlet is in fluid communication with the steam inlet of the steam condenser, such that, in operation, steam generated in the steam generator is passed to the steam condenser to heat the process waste gas stream entering the process waste gas stream inlet. By providing a steam generator linked to the steam condenser the apparatus can advantageously be used to transfer heat from a combusted process waste gas stream exiting the catalyst bed to an incoming process waste gas stream that is to be fed to the catalyst bed. An advantage of carrying out that heat transfer via a steam generator and a steam condenser is that the pressure drop on the process side of the emissions control system can be kept low whilst still retaining efficient heat transfer through the design of the steam side of the emissions control system. Moreover, extra steam can be added, or steam can be removed, as necessary to balance the required heat transfer. For that reason, it may be that the steam outlet of the steam generator is also in fluid communication with a connector for connecting to a steam network, for example a plant steam network.

Preferably the emissions control system comprises a vessel containing the steam condenser, the catalyst bed and the steam generator. Combining all three stages in a single vessel advantageously reduces the cost of the equipment and keeps pressure drops below. Such a combination may also remove the need for high temperature, for example 600° C., flange connections between vessels. The temperature between the catalyst bed and the steam generator may be in the region of 600° C., but if those components are in the same vessel, the only connection needed is that downstream of the steam generator, where the temperature may for example be in the region of 230° C.-245° C.

Preferably the steam generator is located above the catalyst bed. In that way, the process waste gas stream flows up through all parts of the emissions control system one after the other, thus avoiding bends or other significant direction changes that may increase pressure drop.

Preferably the emissions control system further comprises a turbocharger having a turbine side inlet in fluid communication with the catalyst bed and a turbine side outlet in fluid communication with the tube side of the steam generator such that, in operation, the process waste gas stream leaving the catalyst bed is passed to the tube side of the steam generator via a turbine side of the turbocharger. Thus, the energy in the process waste stream can be used to drive the turbine in the turbocharger to recover some of the energy in the process waste stream. The turbocharger may, for example, be configured to pressurise a stream, for example an air stream, fed to the process, thus reducing the new energy required for pressurising the feed stream.

Preferably the emissions control system is for use in a process according to the first aspect of the invention. Desirably, the emissions control system is suitable for retrofitting to existing processes or plants for the production of formaldehyde. Fitting an emissions control system according to the invention may help a plant or process achieve better environmental performance without adversely affecting the pressure drop of the process as a whole.

Preferably the emissions control system is used to treat the waste gas stream in the process.

It will be appreciated that features described in relation to one aspect of the invention may be equally applicable to other aspects of the invention. For example, features described in relation to a process of the invention for the production of formaldehyde may be equally applicable to an emissions control system of the invention and vice versa. It will also be appreciated that optional features may not apply, and may be excluded from, certain aspects of the invention.

### DESCRIPTION OF THE DRAWINGS

The invention will be further described by way of example only with reference to the following figures, of which:

FIG. 1 is a diagram of a prior art Formox process for the production of formaldehyde;

FIG. 2 is a diagram of a process for the production of formaldehyde according to an embodiment of the present invention;

FIG. 3 is an emissions control system according to an embodiment of the invention;

FIG. 4 is an emissions control system according to another embodiment of the invention;

FIG. 5 is an emissions control system according to another embodiment of the invention; and

FIG. 6 is an emissions control system according to another embodiment of the invention.

### DETAILED DESCRIPTION

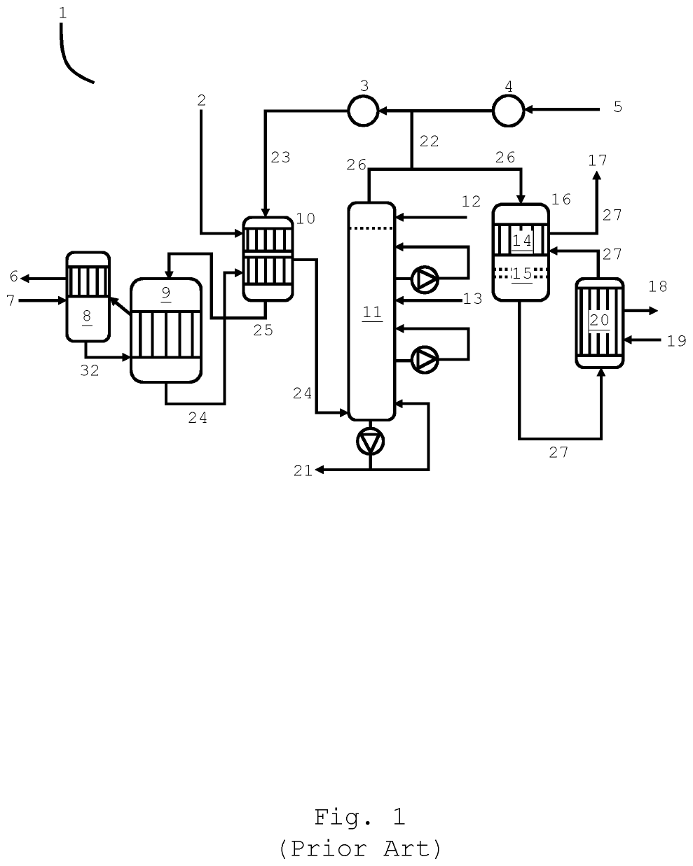

In a prior art Formox process 1 for producing formaldehyde in FIG. 1 a fresh air stream 5 is passed through a pressurisation blower 4 and then mixed with a recirculation stream 22 to form a mixed stream 23 before being fed via a recirculation blower 3 to a vaporiser 10. In the vaporiser 10, the mixed stream 23 is mixed with a methanol stream 2 and vaporised using heat from a process stream 24 leaving a reactor 9. The resulting feed stream 25 is fed to the reactor 9 which, in this embodiment, is an isothermal reactor cooled by vaporisation of a heat transfer fluid 32. The heat transfer fluid 32 passes to a condenser 8, where it is condensed and steam 6 generated from boiler feed water 7, before returning to the reactor 9. In the reactor 9, the methanol in the feed stream 25 reacts on an iron/molybdenum oxide catalyst to produce formaldehyde, which exits the reactor 9 in a process stream 24 comprising the formaldehyde and unreacted parts of the feed stream 25. The process stream 24 passes through the vaporiser 10, where heat in the process stream 24 is used to vaporise the feed stream 25, and is fed to an absorber 11. In the absorber 11, process water 12 and optionally urea 13 flows down and strips the formaldehyde from the process stream 24 flowing up the absorber 11. The water 12, and optionally urea 13, together with the formaldehyde exits the bottom of the absorber as a product stream 21. That product stream 21 is typically 55% formalin, if just process water 12 is used, or UFC if urea 13 is used. The remainder of the process stream 24 exits the top of the absorber as a waste gas stream 26. That waste gas stream 26 is partially recycled as the recirculation stream 22 and the remainder is sent to an emissions control system 16. In the emissions control system 16, the waste gas stream 26 is first heated in a pre-heater 14 using energy from the combusted waste gas stream 27 leaving the emissions control system 16 and then combusted in a catalyst bed 15 having a catalyst comprising PPd and PPt to form the combusted waste gas stream 27. The combusted waste gas stream 27 leaving the catalyst bed 15 has a temperature of around 500° C. to 550° C. and is fed to a steam generator 20, where the combusted waste gas stream 27 is cooled and boiler feed water 19 is turned into steam 18, and then fed back to the pre-heater 14 of the emissions control system 16 to heat the incoming waste gas stream 26. The combusted waste gas stream 27 leaving the pre-heater 16 is sent to a stack 17.

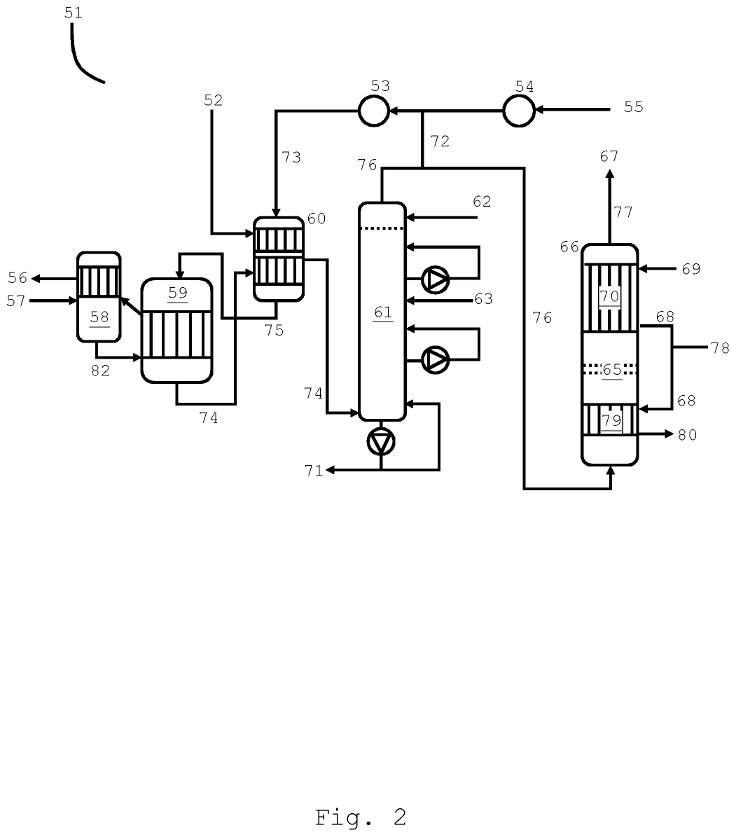

In FIG. 2 a process according to the invention is presented. A fresh air stream 55 is passed through a pressurisation blower 54 and then mixed with a recirculation stream 72 to form a mixed stream 73 before being fed via a recirculation blower 53 to a vaporiser 60. In the vaporiser 60, the mixed stream 73 is mixed with a methanol stream 52 and vaporised using heat from a process stream 74 leaving a reactor 59. The resulting feed stream 75 is fed to the reactor 59 which, in this embodiment, is an isothermal reactor cooled by vaporisation of a heat transfer fluid 82. The heat transfer fluid 82 passes to a condenser 58, where it is condensed and steam 56 generated from boiler feed water 57, before returning to the reactor 59. In the reactor 59, the methanol in the feed stream 75 reacts on an iron/molybdenum oxide catalyst to produce formaldehyde, which exits the reactor 59 in a process stream 74 comprising the formaldehyde and unreacted parts of the feed stream 75. The process stream 74 passes through the vaporiser 60, where heat in the process stream 74 is used to vaporise the feed stream 75, and is fed to an absorber 61. In the absorber 61, process water 62 and optionally urea 63 flows down and strips the formaldehyde from the process stream 74 flowing up the absorber 61. The water 62, and optionally urea 63, together with the formaldehyde exits the bottom of the absorber as a product stream 71. That product stream 71 is typically 55% formalin, if just process water 62 is used, or UFC if urea 63 is used. The remainder of the process stream 74 exits the top of the absorber as a waste gas stream 76. That waste gas stream 76 is partially recycled as the recirculation stream 72 and the remainder is sent to an emissions control system 66. In the emissions control system 66, the waste gas stream 76 is first heated in a steam condenser 79. The waste gas stream 76 flows in to the bottom of the steam condenser 79 and up through the condenser 79. Steam 68 entering the steam condenser 79 condenses on the tubes and flows down and out of the steam condenser 79 as condensate 80. The condensate 80 is collected and re-used. The heated waste gas stream thus created flows from the steam condenser 79 to the catalyst bed 65 having a catalyst comprising PPd and PPt. In the catalyst bed 65 components of the heated waste gas stream such as carbon monoxide, DME and methanol are combusted to form a combusted waste gas stream, which enters a steam generator 70. In the steam generator 70 the combusted waste gas stream is cooled and boiler feed water 69 is turned into steam 68. The steam 68 may be 12 barg steam, which coincides with the minimum export steam pressure from a standard plant. The steam 68 raised in the steam generator 70 is fed to the steam condenser 79 to raise the temperature of the incoming waste gas stream 76. The steam 68 can also be fed to, or supplemented from, the plant steam network 78. The combusted waste gas stream 77 exiting the steam generator 70 is sent to a stack 67. The stack 67 temperature depends on the pressure of the steam 68. For example, with a temperature approach (that is, the temperature difference between the combusted waste gas stream 77 and the steam) of 25° C. a stack 67 temperature of 225° C. corresponds to a steam 68 pressure of 14.6 barg and a stack 67 temperature of 245° C. corresponds to a steam 68 pressure of 22.2 barg. The steam condenser 79, catalyst bed 65 and steam generator 70 are all contained within a single vessel. The flanges and piping at the vessel exit need to be suitable to handle the stack 67 temperature, which is significantly lower than the 500° C.-550° C. that the connections between the emissions control system 16 and the steam generator 20 in the prior art process 1 of FIG. 1 need to handle. Advantageously, this may even permit higher process temperatures, for example 600° C., to be used at the exit of the catalyst bed 65, since, unlike the prior art, there is no need for pipework and flanges at the exit of the catalyst bed 65 when the steam condenser 79, catalyst bed 65 and steam generator 70 are all contained within a single vessel.

During start-up steam from elsewhere in the plant steam network 78, can be fed to the steam condenser 79, thus removing the need for a separate electrical heater for the emissions control system 66.

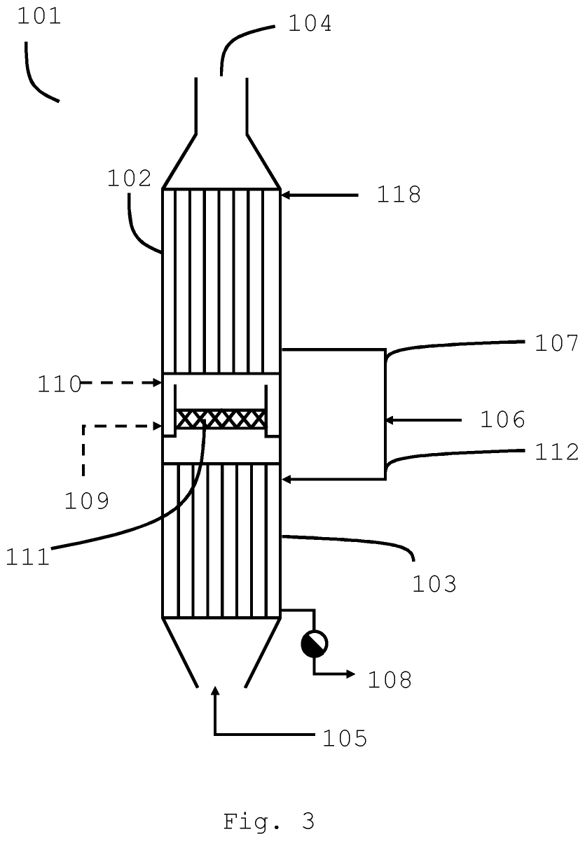

In FIG. 3 an emissions control system 101 is provided for the catalytic combustion of components of a process waste gas stream 105. The emissions control system 101 comprises a catalyst bed 111 comprising a catalyst for the catalytic combustion of the components of the process waste gas stream 105. The catalyst typically comprises PPd and PPt, for example as supplied by Johnson Matthey Formox. A steam condenser 103 has a tube side in fluid communication with a process waste gas stream inlet, where the process waste gas stream 105 is fed to the emissions control system 101, and with the catalyst bed 111. The steam condenser 103 has a shell side in fluid communication with a steam inlet fed from a steam stream 112 and a condensate outlet 108. Downstream of the catalyst bed 111 the emissions control system 101 further comprises a steam generator 102 having a tube side in fluid communication with the catalyst bed 111 and a process waste gas stream outlet 104, and a shell side in fluid communication with a boiler feed water inlet 118 and a steam outlet 107. The steam outlet 107 is in fluid communication with the steam inlet stream 112 of the steam condenser 103. A steam stream 106 connects with the steam outlet 107 and the steam inlet stream 112 so that excess steam can be removed or make-up steam added as required at any particular time.

The steam condenser 103, catalyst bed 111 and steam generator 102 are in a single vessel. The outlet temperature of the vessel is around 225° C.-245° C., which is significantly cooler than the 500° C.-550° C. temperature of the combusted waste gas stream leaving the catalyst bed 111. By feeding that stream straight from the catalyst bed 111 to the steam generator 102 in the same vessel, the need for high temperature piping and connections is removed. The removal of piping and connections in the high temperature region downstream of the catalyst bed 111 may allow higher process temperature, for example 600° C., to be used at that point in the process.

The steam condenser 103 is at the bottom of the vessel, with the catalyst bed 111 above it and the steam generator 102 above that. In operation, the process waste gas stream leaving the catalyst bed 111 is cooled in the steam generator 102 before exiting the process waste gas stream outlet 104 and steam generated in the steam generator 102 is passed to the steam condenser 103 to heat the process waste gas stream 105 entering the process waste gas stream inlet. Chill gas 109, which might for example be air at ambient temperature, or steam 110 for heating can also be fed to the emissions control system 101 to further control the temperature if required. The process waste gas stream 105 flows upwards through the emissions control system 101, with steam stream 112 fed to the top of the steam condenser 103 and condensate removed from the condensate outlet 108 at the bottom of the steam condenser 103. Steam condensing on the outside of the tubes of the steam condenser 103 will thus flow downwards under gravity toward the condensate outlet 108. The process waste gas stream 105 enters the bottom of the emissions control system 101 and flows in a relatively straight path up through the emissions control system 101, thus avoiding unnecessary pressure drops. Compression costs may be significant in formaldehyde production and any pressure drops, even in the emissions control system 101, must be accounted for in the initial compression of the feed gases. Avoiding unnecessary pressure drops may therefore be important for producing a cost-efficient process.

In operation, the incoming process waste gas stream 105 is thus heated by the condensing steam in the steam condenser 103 before being combusted in the catalyst bed 111. The hot combusted waste gas stream leaving the catalyst bed 111 is cooled in the steam generator 102, generating steam 107 that is in turn used to run the steam condenser 103. The heat transfer efficiency on the steam side of the steam generator 102 and steam condenser 103 can be optimised without affecting the pressure drop of the process side, unlike in prior art systems where heat is transferred directly between the outgoing combusted waste gas stream and the incoming process waste gas stream. When the steam generated in the steam generator 102 is not sufficient to pre-heat the incoming process waste gas stream 105, for example during start up, the steam condenser 103 can be fed with steam from another part of the plant via steam stream 106. That removes the need for a dedicated heater for start-up of the emissions control system 101, thus saving on capital costs.

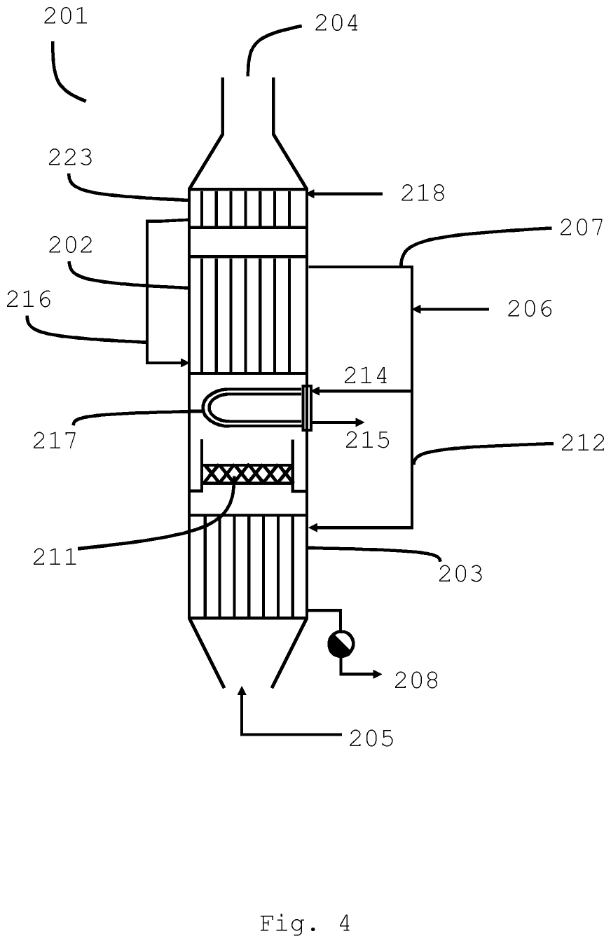

In FIG. 4, an emissions control system 201 is fed with a process waste gas stream 205. At the upstream end of the emissions control system 201, which is at the bottom of the vessel in which the emissions control system is contained in FIG. 4, there is a steam condenser 203. The tube side of the steam condenser 203 is in fluid communication with the process waste gas stream 205 and the catalyst bed 211. The process waste gas stream 205 flows up through the steam condenser 203 and through the catalyst bed 211 where hazardous components of the stream are combusted to form a combusted waste gas stream.

Downstream of the catalyst bed 211 there is a steam superheater 217. Downstream of the steam superheater 217 is a steam generator 202 and an economiser 223. The shell side of the economiser 223 is fed with boiler feed water 218 and has an outlet stream 216 which connects to a shell side inlet of the steam generator 202. The shell side of the steam generator 202 has an outlet steam stream 207, which connects with a steam stream 206 by which steam can either be removed or added as necessary. After the connection, the steam stream splits to a stream 214 that feeds to the steam superheater 217 to create superheated export steam 215 and to a steam stream 212 that is fed to the steam condenser 203. The combusted waste gas stream leaving the catalyst bed 211 passes through the shell side of the steam superheater 217, through the tube side of the steam generator 202 and then through the tube side of the economiser 223 before exiting through the combusted gas stream outlet 204, which is typically fed to a stack.

As with the embodiment in FIG. 3, the process waste gas stream 205 is heated in the steam condenser 203 before being combusted in the catalyst bed 211 to combust hazardous components and create a combusted waste gas stream. The combusted waste gas stream is then cooled in the steam superheater 217, steam generator 202 and economiser 223. The economiser 223 may be replaced with a low-pressure steam generator. The economiser 223 or low-pressure steam generator improve the heat recovery efficiency by making use of the low temperature heat remaining in the combusted waste gas stream after it has passed through the steam generator 202. Boiler feed water 218 fed to the shell side of the economiser 223 is heated by the cooling of the combusted waste gas stream and fed to the shell side of the steam generator 202 where it is turned into steam. The steam is fed to the steam superheater 217 to create superheated steam 215 for export to other parts of the plant or to the steam condenser 203 to pre-heat the incoming process waste gas stream 205. Again, as with the embodiment in FIG. 3, the emissions control system 201 can be started using steam from elsewhere in the plant via steam stream 206, thus removing the need for a dedicated start-up heater. Moreover, the heat transfer efficiency on the steam side of the emissions control system 201 can be optimised without affecting the pressure drop of the process side.

Again, the emissions control system 201 is contained in a single vessel. That may be advantageous as it reduces the need for inter-vessel connections, and particularly high-temperature inter-vessel connections. That may reduce capital costs and also pressure drops, which may in turn reduce operating costs. Because the steam condenser 203 is at the bottom of the vessel and the process waste gas stream flows up from the steam condenser 203 through the catalyst bed 211, the support net on which the catalyst bed rests is at the cooler end of the catalyst bed 211. That may be advantageous since a support net of sufficient strength may be more readily provided when it does not have to withstand the high temperatures at the exit of the catalyst bed 211. A secondary net may be provided above the catalyst bed 211 to prevent catalyst being carried away in the combusted waste gas stream, but that net does not need to support the full weight of the catalyst bed 211.

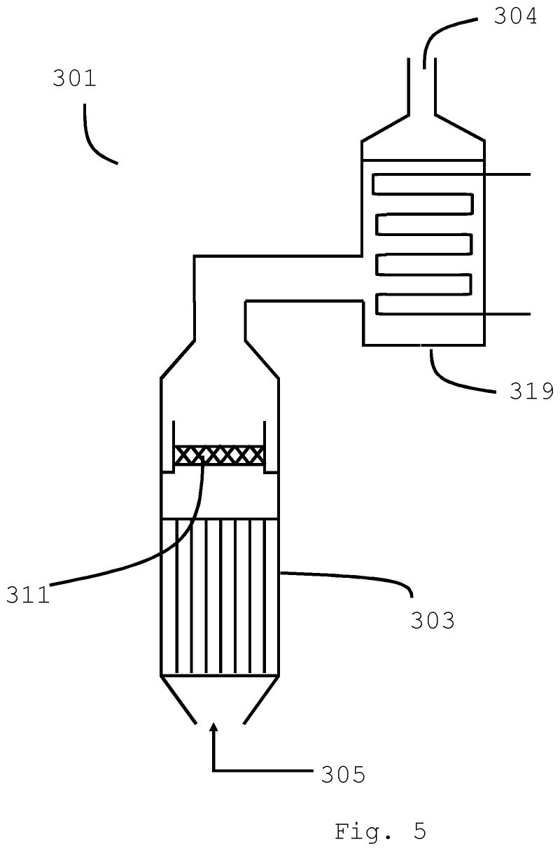

In FIG. 5 an emissions control system 301 comprises a steam condenser 303, a catalyst bed 311 and a furnace-type steam super heater 319. The furnace-type steam super heater 319 may be used to generate super-heated steam. Producing super-heated steam in this way may increase the stack temperature as it is not possible to recover low temperature heat in the furnace-type steam super heater 319. However, it has the advantage of generating super-heated steam, which may be valuable elsewhere on the plant. A process waste gas stream 305 is pre-heated in the steam condenser 303 before passing to the catalyst bed 311 where the hazardous components are combusted to form a combusted waste gas stream. The combusted waste gas stream is fed to the furnace-type steam super heater 319 which generates super-heated steam while cooling the combusted waste gas stream. The cooled combusted waste gas stream exits the furnace-type steam super heater 319 via the outlet 304 and passes to a stack. Super-heated steam raised in the furnace-type steam super heater 319 can be fed to the shell side of the steam condenser 303 to be used in pre-heating the incoming process waste gas stream 305. In this embodiment, the furnace-type steam super heater 319 is in a different vessel to the vessel containing the steam condenser 303 and the catalyst bed 311. While there may be advantages, for example in terms of reduced connections and hence reduced pressure drops, by having everything in one vessel, there may be occasions when it is preferable to use more than one vessel, for example due to space constraints when upgrading an existing process.

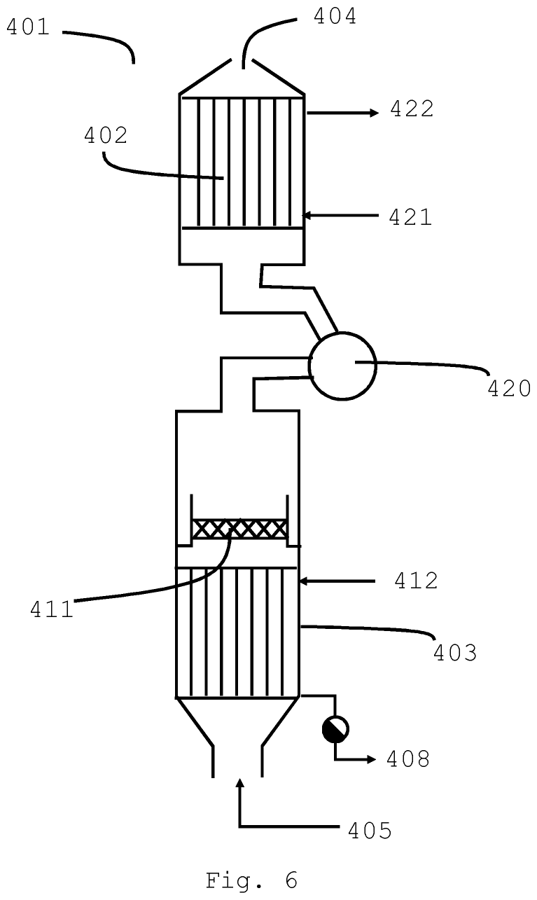

In the emissions control system 401 of FIG. 6 a catalyst bed 411 is located downstream of, and in this embodiment above, a steam condenser 403. A process waste gas stream 405 flows up through the tube side of the steam condenser 403 and then up through the catalyst bed 411. As explained above in relation to other embodiments, flowing the process waste gas stream 405 up through the catalyst bed 411 provides advantages in terms of the temperature conditions to which the support net for the catalyst bed 411 is exposed. The steam condenser 403 is fed with steam from a steam inlet stream 412 near the top of the shell side and condensate exits through a condensate outlet 408 near the bottom of the shell side. Thus, the steam condenses on the tubes and flows down under gravity to the condensate outlet 408. In doing so it heats the process waste gas stream 405 before it is fed to the catalyst bed 411.

The combusted waste gas stream leaving the catalyst bed 411 is fed to a turbocharger 420. In the turbocharger 420 the pressure of the combusted waste gas stream is reduced and a feed stream to the process is pressurised. Typically, the combusted waste gas stream passes through the expander part of the turbocharger 420, and a fresh air feed stream to the process passes through the compressor part of the turbocharger 420. Compression of process gases may be a significant operating cost in a formaldehyde production process and recovering some of the energy in the combusted waste gas stream as compression of a feed stream may therefore be advantageous.

From the turbocharger 420 the combusted waste gas stream passes through the tube side of a steam generator 402, which is fed with boiler feed water 421 on the shell side to raise steam 422. The steam thus raised is fed to the steam inlet stream 412, either with withdrawal or addition of further steam as necessary, and used to pre-heat the incoming process waste gas stream 405. Thus, the energy in the combusted waste gas stream is used to pre-heat the incoming process waste gas stream 405, but the heat is transferred indirectly using the steam generator 402 and steam condenser 403. As discussed above, that has several advantages including the opportunity to reduce pressure drops for the process waste gas stream and to use substitute steam from another part of the plant during start-up, thus removing the need for a dedicated start-up heater for the emissions control system 401. Including the turbocharger 420 permits the energy in the combusted waste gas stream to be used effectively by using it in the turbocharger 420 while the combusted waste gas stream is at its hottest and then using it to generate steam in the steam generator 402 after it has passed through the turbocharger 420.

The emissions control systems 101, 201, 301, 401 of FIGS. 3, 4, 5 and 6, could be used, for example, in the process 51 of FIG. 2.

It will be appreciated that the embodiments set out above are examples of the invention and that the skilled person would appreciate that variations were possible within the scope of the invention. For example, the steam condenser and steam generator may be in the same or different vessels and the system could be arranged horizontally or with side-by-side vessels. The process waste gas stream may flow down or horizontally through some or all parts of the process.

1. A process for the production of formaldehyde, the process comprising:
a. feeding a feed stream comprising methanol to a reactor;
b. converting the methanol to formaldehyde in the reactor using a mixed oxide catalyst to produce a process stream comprising formaldehyde;
c. separating formaldehyde from the process stream to create a product stream comprising formaldehyde and a waste gas stream;
d. feeding at least part of the waste gas stream to a steam condenser to raise the temperature of the at least part of the waste gas stream to create a heated waste gas stream; and
e. feeding the heated waste gas stream to a catalytic combustion bed to catalytically combust components of the heated waste gas stream to create a combusted waste gas stream.

2. A process according to claim 1, wherein the steam condenser and the catalyst bed are contained within a single vessel.

3. A process according to claim 1, wherein the steam condenser is a shell and tube steam condenser and the waste gas stream flows through the tube side of the steam condenser and steam condenses in the shell side of the steam condenser.

4. A process according to claim 1, wherein the process further comprises:
f. Feeding the combusted waste gas stream to a steam generator wherein the combusted waste gas stream is cooled and steam is produced.

5. A process according to claim 4, wherein the steam generator is a shell and tube steam generator and the combusted waste gas stream flows through the tube side of the steam generator and steam is generated in the shell side of the steam generator.

6. A process according to claim 4, wherein, before being fed to the steam generator, the combusted waste gas stream is fed through an expander part of a turbocharger to drive a compressor part of the turbocharger in order to pressurise an air stream fed to the process to form part of the feed stream.

7. A process according to claim 4, wherein the process further comprises:
g. Feeding steam from the steam generator to the steam condenser to raise the temperature of the waste gas stream in step d.

8. A process according to claim 7, wherein the steam condenser, the catalyst bed and the steam generator are contained within a single vessel.

9. An emissions control system for the catalytic combustion of components of a process waste gas stream, the emissions control system comprising: a catalyst bed comprising a catalyst for the catalytic combustion of the components of the process waste gas stream; and a steam condenser having a tube side in fluid communication with a process waste gas stream inlet and the catalyst bed, and a shell side in fluid communication with a steam inlet and a condensate outlet, such that, in operation, a process waste gas stream entering the process waste gas stream inlet is heated in the steam condenser before passing to the catalyst bed.

10. An emissions control system according to claim 9, wherein the emissions control system comprises a vessel containing both the catalyst bed and the steam condenser.

11. An emissions control system according to claim 9 wherein the emissions control system further comprises a steam generator having a tube side in fluid communication with the catalyst bed and a process waste gas stream outlet, and a shell side in fluid communication with a boiler feed water inlet and a steam outlet, such that, in operation, the process waste gas stream leaving the catalyst bed is cooled in the steam generator, converting boiler feed water entering through the boiler feed water inlet into steam exiting through the steam outlet, before exiting the process waste gas stream outlet.

12. An emissions control system according to claim 11, wherein the emissions control system comprises a vessel containing the steam condenser, the catalyst bed and the steam generator.

13. An emissions control system according to claim 11, wherein the emissions control system further comprises a turbocharger having an expander side inlet in fluid communication with the catalyst bed and an expander side outlet in fluid communication with the tube side of the steam generator such that, in operation, the process waste gas stream leaving the catalyst bed is passed to the tube side of the steam generator via an expander side of the turbocharger.

14. An emissions control system according to claim 11, wherein the steam outlet is in fluid communication with the steam inlet of the steam condenser, such that, in operation, steam generated in the steam generator is passed to the steam condenser to heat the process waste gas stream entering the process waste gas stream inlet.

15. An emissions control system for the catalytic combustion of components of a process waste gas stream, the emissions control system comprising: a catalyst bed comprising a catalyst for the catalytic combustion of the components of the process waste gas stream; and a steam condenser having a tube side in fluid communication with a process waste gas stream inlet and the catalyst bed, and a shell side in fluid communication with a steam inlet and a condensate outlet, such that, in operation, a process waste gas stream entering the process waste gas stream inlet is heated in the steam condenser before passing to the catalyst bed, wherein the emissions control system is for use in a process according to claim 1.

16. Use of an emissions control system for the catalytic combustion of components of a process waste gas stream, the emissions control system comprising: a catalyst bed comprising a catalyst for the catalytic combustion of the components of the process waste gas stream; and a steam condenser having a tube side in fluid communication with a process waste gas stream inlet and the catalyst bed, and a shell side in fluid communication with a steam inlet and a condensate outlet, such that, in operation, a process waste gas stream entering the process waste gas stream inlet is heated in the steam condenser before passing to the catalyst bed to treat the waste gas stream in a process according to claim 1.
